(19) United States
(12) Patent Application Publication
(10) Pub. No.: US 2012/0130342 A1
(43) Pub. Date: May 24, 2012

(54) AUTOINJECTOR HAVING AN OUTER PACKAGING AND A NEEDLE SHEATH REMOVING MEANS

(75) Inventor: Ian Charles Cleathero, Melton Mowbray (GB)

(73) Assignee: THE MEDICAL HOUSE LIMITED, South Yorkshire (GB)

(21) Appl. No.: 13/147,568

(22) PCT Filed: Feb. 2, 2010
(86) PCT No.: PCT/GB10/50161

§ 371 Date: Feb. 10, 2012

(30) Foreign Application Priority Data
Feb. 5, 2009 (GB) 0901801.1

(51) Int. Cl. A61M 5/32

(52) U.S. Cl. 604/506; 206/365

(57) ABSTRACT

An autoinjector device for delivering a dose of medicament including a syringe including a needle wherein, prior to use of the device, the needle is substantially covered by a needle sheath (107). The autoinjector further includes a housing (112) in which said syringe is located, an outer packaging in which said housing is located, where the outer packaging comprising a front packaging (101) part and a rear packaging part. The autoinjector also includes needle sheath removing means (103) associated with said front packaging part (101), wherein, prior to use of the device, said front and rear packaging parts cooperate together to entirely enclose said housing, syringe and needle sheath, and wherein, upon axial separation of said front and rear packaging parts, said needle sheath removing means is capable of causing axial separation of said needle sheath from said needle so as to remove said needle sheath therefrom.

This invention relates to the field of autoinjectors for the administration of liquid medication, for example, adrenaline (epinephrine) for the treatment of anaphylaxis.

### BACKGROUND

An autoinjector is an automatic injection device designed to facilitate automated delivery of a dose of medicament to a patient through a hypodermic needle, the injection usually being administered by the patient themselves. An autoinjector works, for example, by delivering an injection automatically upon actuation by the patient pressing a button, moving a lever or part of a housing etc. This is in contrast to a conventional manual syringe where the patient himself needs to directly depress a plunger into a barrel containing medicament in order to effect the injection. The terms “autoinjector” and “injection device” are used interchangeably in the following description.

Examples of autoinjectors are described in WO2003/099358 (Seedlings Life Science Ventures LLC) and WO01/93926 (Mayo Foundation for Medical Education and Research). These are both generally flat devices which are of small size to encourage users to carry the device with them for ready access. GB2396298 (PA Consulting Services Ltd) is an example of a more conventionally-shaped elongate autoinjector, but of relatively complex internal construction.

A well-known use for autoinjectors is the delivery of adrenaline (epinephrine) for the emergency treatment of severe allergic reactions or anaphylaxis. Examples of autoinjectors of this type are marketed under the trade mark EPIPEN, see www.epipen.co.uk or ANAPEN, see www.anapen.co.uk. These devices deliver a single dose of epinephrine intramuscularly, usually into the patient's thigh and will be referred to generally below as epinephrine autoinjectors (although these devices can equally well be used to deliver other medicaments). After delivery of medicament, the user pulls the needle from the tissue as the device is moved away from the injection site. In some embodiments, a needle cover may move forward to conceal the needle as it is withdrawn in order to minimise the risk of needle-stick injury (see for example U.S. Pat. No. 7,449,012 Meridian Medical Technologies).

Most of the above prior art devices have a custom designed medicament chamber therein rather than being built around a standard pre-filled syringe presentation. The custom medicament chamber, although allowing for a compact overall size for the device, means that the device as whole must be subjected to more rigorous regulatory control as compared with a device containing a standard pre-filled syringe presentation which will have already obtained regulatory approval.

An improved autoinjector is described in our international patent application, published under number WO 2005/070481, and in later patent applications, which has the significant advantages, inter alia, that it can be built around a conventional or standard syringe presentation and that the needle is automatically withdrawn after delivery of medicament and is never in sight of the patient. Such autoinjectors will be referred to generally below as “ASI autoinjectors”. ASI is a trade mark of The Medical House plc. Owing to the different internal construction and operation of an ASI autoinjector, the user may find it less easy to use than the more familiar devices such as the EPIPEN or ANAPEN epinephrine autoinjectors, that he/she may already be practised at using. It would therefore be advantageous to provide an improved ASI autoinjector based around the technology disclosed in WO 2005/070481 but having an external appearance and user-operated steps comparable to the more familiar devices.

### SUMMARY OF THE INVENTION

In accordance with a first aspect of the present invention, there is provided an autoinjector device for delivering a dose of medicament including:
a syringe including a needle wherein, prior to use of the device, the needle is substantially covered by a needle sheath;
a housing in which said syringe is located;
an outer packaging in which said housing is located, the outer packaging comprising a front packaging part and a rear packaging part; and
needle sheath removing means associated with said front packaging part,
wherein, prior to use of the device, said front and rear packaging parts cooperate together to entirely enclose said housing, syringe and needle sheath, and
wherein, upon axial separation of said front and rear packaging parts, said needle sheath removing means is capable of causing axial separation of said needle sheath from said needle so as to remove said needle sheath therefrom.

Preferably said housing and said front packaging part include axial guide elements that cooperate to limit the degree of relative rotation therebetween. Further preferably, said axial guide elements include axial ribs on one or both of said housing and said front packaging part.

In one preferable embodiment, said needle sheath removing means includes gripping elements that are capable of radially gripping said needle sheath. Said gripping elements preferably include a plurality of radially moveable, axially extending fingers, each of said plurality of fingers having an enlarged head capable of radially gripping said needle sheath. Furthermore, each of said enlarged heads preferably comprises a cam surface on a radially outward surface and said housing comprises an axially extending boss positioned to interfere with said cam surfaces during axial separation, wherein interference of said cam surfaces with said boss causes said plurality of fingers to flex further radially inwardly to grip said needle sheath.

In another preferable embodiment, the autoinjector further comprises a priming means which is required to be primed in order to ready the device for delivery of medicament. Said priming means is preferably a safety clip that prevents actuation and said priming involves removing the safety clip from the device. Said safety clip is preferably attachable to a rear end of said housing and comprises an axially extending spigot that is capable of interfering with a trigger mechanism of the device to prevent actuation. Alternatively, said priming means is preferably an actuation button, and said priming involves pressing said button.

In one preferable embodiment said axial separation involves the axial pulling of said front packaging part relative to said rear packaging part. Preferably, said front packaging part is releasably attachable to said rear packaging part by push fit or snap fit engagement.

In an alternative preferable embodiment, said front packaging part and said rear packaging part have complementary screw threads and, prior to use, said front and rear packaging parts cooperate together by engagement of said complementary screw threads, wherein said axial separation involves the unscrewing of said front packaging part relative to said rear packaging part. Preferably, said housing is axially and rotationally fixable with respect to said rear packaging part during axial separation of said front packaging part relative to said rear packaging part up to an axial separation threshold. Further preferably, the autoinjector further comprises a cap insert that is axially and rotationally fixable relative to said rear packaging part, wherein said housing is axially and rotationally fixable with respect to said rear packaging part up to said axial separation threshold by engagement of said housing with said cap insert. Preferably, said cap insert is arranged in said rear packaging part to define an annular gap that is adapted to receive a portion of the front packaging part;
wherein, during axial separation of said front packaging part and said rear packaging part up to said axial separation threshold, said cap insert is radially engagable with said housing to prevent relative axial displacement between said housing and said cap insert, and said portion of said front packaging part is capable of preventing radial disengagement of the cap insert from the housing;
and, during axial separation of said front packaging part relative to said rear packaging part beyond said axial separation threshold, said cap insert is radially disengageable from said housing.

Said cap insert preferably comprises radially flexible panels each having a plurality of circumferential ribs projecting radially inwardly therefrom, wherein the plurality of ribs are engageable with a plurality of circumferential grooves on said housing.

In accordance with a second aspect of the present invention, there is provided a method of preparing an autoinjector device for delivering a dose of medicament comprising the steps of:
providing an autoinjector device in accordance with the first aspect of the present invention;
axially separating said front and rear packaging parts, causing said needle sheath to be removed from said needle; and
removing said front and rear packaging parts from the device;
such that the autoinjector device is ready to deliver a dose of medicament.

Preferably, the method further comprises the step of priming the device using said priming means such that autoinjector device is ready to deliver a dose of medicament.

In one embodiment, the step of axially separating said front and rear packaging parts preferably includes axially pulling the front packaging forwardly relative to said rear packaging part, consequently causing said needle sheath to be removed from said needle.

In an alternative preferable embodiment, the step of axially separating said front and rear packaging parts includes unscrewing said front packaging part from said rear packaging part, consequently causing said needle sheath to be removed from said needle. Further preferably, the step of axially separating said front and rear packaging parts includes unscrewing said front packaging part from said rear packaging part at least up to said axial separation threshold, and then axially pulling said front packaging part from said rear packaging part.

### BRIEF DESCRIPTION OF THE DRAWINGS

Preferred embodiments of the present invention will now be more particularly described, by way of example only, with reference to the accompanying drawings in which:

FIG. 1 is a perspective view of an autoinjector in its packaging;

FIG. 2 is a cross-sectional view of the FIG. 1 autoinjector;

FIG. 3 shows the autoinjector with the rear part of the packaging removed;

FIG. 4 is a cross-sectional view of the FIG. 3 autoinjector;

FIG. 5 shows more detail of the front end of the front part of the packaging;

FIG. 6 shows how the front end of the front part of the packaging interacts with the front end of the medicament-delivering part of the autoinjector;

FIG. 7 is a perspective view of the medicament-delivering part of the autoinjector, removed from the packaging but with the safety clip still in place;

FIG. 8 shows the autoinjector of FIG. 7 with the safety clip removed;

FIG. 9 is a cross-sectional view of the autoinjector of FIG. 8, ready to deliver medicament;

FIG. 10 is perspective view of an alternative autoinjector in its packaging;

FIG. 11 is a cross-sectional view of the rear end of the autoinjector of FIG. 10, prior to removal of the rear part of the packaging;

FIG. 12 is a perspective view of the cap insert of the autoinjector of FIGS. 10 and 11;

FIG. 13 is a cross-sectional view of the rear end of the autoinjector of FIGS. 10-12, during removal of the rear part of the packaging;

FIG. 14 is a cross-sectional perspective view of the front end of the forward part of the packaging of FIGS. 10 to 13;

FIG. 15 is a cross-sectional view of the front end of the autoinjector of FIGS. 10 to 14, prior to removal of the needle sheath;

FIG. 16 is a cross-sectional view of the front end of the autoinjector of FIGS. 10 to 15, during removal of the needle sheath;

FIG. 17A is a perspective view of the front end of the autoinjector of FIGS. 10 to 16 that shows the front housing;

FIG. 17B is a transparent perspective view of the front end of the front part of the packaging;

FIG. 18 shows a cross-sectional view of the rear end of the autoinjector of FIGS. 10 to 17B prior to removal of the safety clip;

FIG. 19 shows a cross-sectional view of the rear end of the autoinjector of FIGS. 10 to 18 with the safety clip removed; and

FIG. 20 shows a perspective view of the outer housing of the autoinjector of FIGS. 10 to 19, as viewed from the front end.

### DETAILED DESCRIPTION

Throughout the description and claims of this specification, the words “comprise” and “contain” and variations of the words, for example “comprising” and “comprises”, means “including but not limited to”, and is not intended to (and does not) exclude other components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Throughout the following description, reference to a “forward” direction means the direction which is towards the patient when the injection device is in use. The “forward” end of the injection device is the end nearest the patient's skin when the device is in use. Similarly, reference to a “rearward” direction means the direction which is away from the patient and the “rearward” end of the device is the end furthest from the patient's skin when the injection device is in use.

Features, integers, characteristics or groups described in conjunction with a particular aspect, embodiment or example of the invention are to be understood to be applicable to any other aspect, embodiment or example described herein unless incompatible therewith.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Conventional epinephrine autoinjectors of the type mentioned above have the following basic user-operated steps:
1. The medicament-delivering part of the autoinjector is provided in a generally tubular two-part outer packaging. Firstly, the rear part of the packaging is opened, removed and discarded.
2. Secondly, the autoinjector is tipped, pulled or withdrawn from the forward part of the packaging, which can then be discarded.
3. Next, a safety clip, catch or cap is removed from the rear end of the autoinjector. The autoinjector cannot be fired while the safety clip is in place.
4. Once the safety clip has been removed, the autoinjector is pushed against the injection site with a downward motion which actuates the automatic delivery of medicament.
5. The user holds the autoinjector at the injection site typically for ten seconds to ensure the dose of medicament is fully delivered, after which the autoinjector can be withdrawn.

In contrast, an ASI autoinjector (for example as described in WO 2005/070481) has the following user-operated steps:
1. No external packaging needs to be removed by the user.
2. The medicament-delivering part of the ASI autoinjector includes a standard syringe whose needle is embedded in and protected by a rubber needle sheath. The needle sheath is optionally surrounded by a rigid needle cover. The autoinjector housing has a front end cap which, when removed by the user, simultaneously removes the needle sheath and needle cover, if present.
3. No safety clip, catch, cap or like needs to be removed by the user from the rear of the autoinjector.
4. Once the front end cap has been removed, the autoinjector is ready to use. Holding the rear end of the outer housing, the user pushes the autoinjector against the injection site with a downward motion to actuate the automatic delivery of medicament.
5. After delivery of medicament, the needle retracts into the housing and the user can remove the autoinjector from the injection site.

A key difference between the two methods is that, in the conventional epinephrine autoinjector, the user needs to remove two sections of outer packaging and then a safety clip from the rear end of the autoinjector before the autoinjector can be actuated to deliver medicament. In an ASI autoinjector, the user does not need to remove any outer packaging and it is the front end cap which serves as the nearest equivalent to a safety clip i.e. the autoinjector cannot be actuated until the front end cap has been removed. The two methods of preparing an autoinjector are therefore significantly different and it may be confusing for a user, likely already to be in a stressed state, to remember which type is which.

Therefore, there is provided an ASI autoinjector which has been modified to make the preparatory method steps as close as possible for the user as those needed for a conventional epinephrine autoinjector.

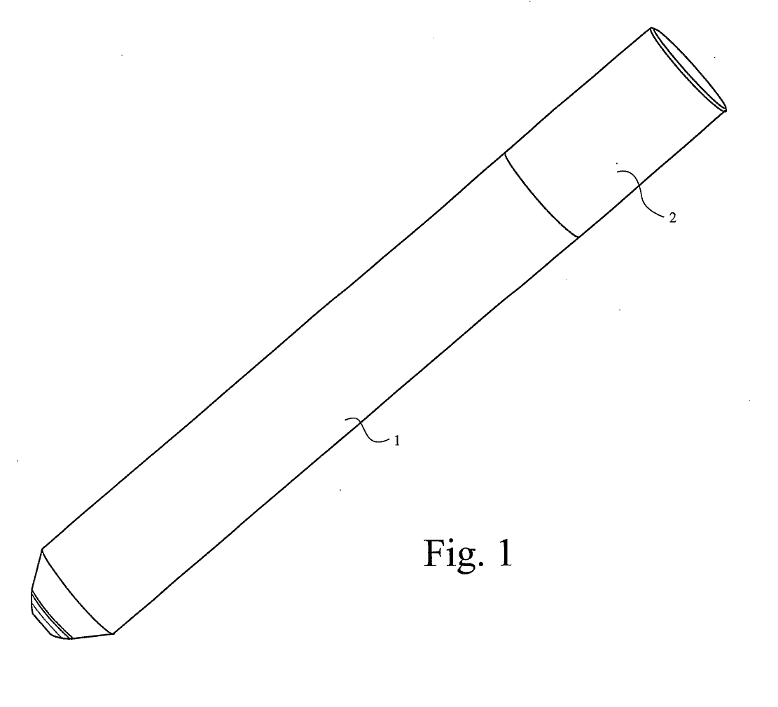

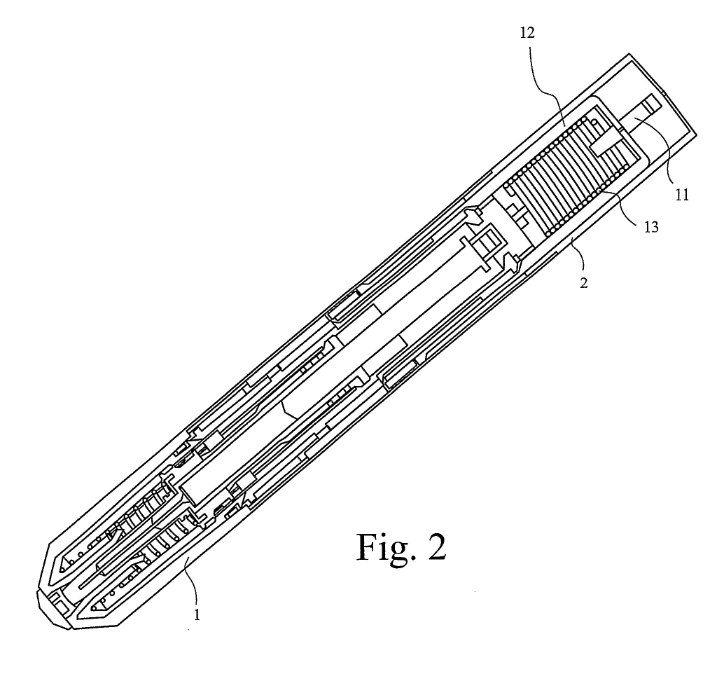

As shown in FIGS. 1 and 2, an autoinjector according to the present invention includes a two-part outer packaging 1, 2. The forward part 1 of the outer packaging is actually an extended version of the front end cap of the ASI autoinjector described in WO 2005/070481 et al and continues to serve the function of preventing premature actuation of the autoinjector in the manner described in our earlier patent applications. The rear part 2 of the outer packaging is generally cylindrical and can snap or push-fit onto the forward part 1 so that the medicament-delivering part of the autoinjector is preferably entirely contained and protected.

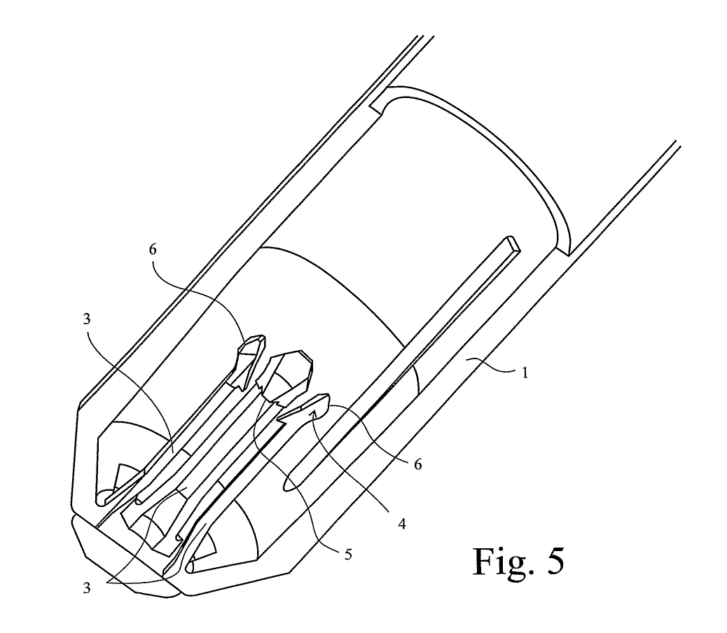

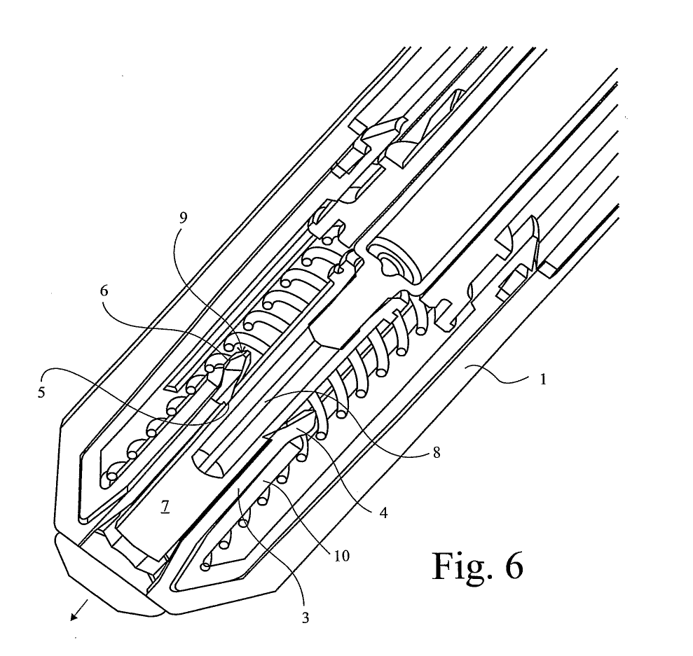

Referring now to FIGS. 5 and 6, the interior of the front end of the forward part of the packaging 1 will be described. The interior front end of the forward part of the packaging 1 is provided with a plurality (preferably four) inwardly axially extending fingers 3 each having an enlarged head or “claw” 4 which is capable of gripping radially inwardly on the rubber needle sheath as shown in FIG. 6. Each claw 4 has an angled blade 5 on its inner surface, and a cam surface 6 on its outer surface.

As shown in FIG. 6, before removal of the forward part 1 of the packaging, the blades 5 are engaged in the rubber needle sheath 7 which surrounds the needle 8 of the standard syringe contained within the autoinjector.

When the user removes the autoinjector from the forward part 1 of the packaging, the packaging moves relative to the needle sheath 7 therein in the direction indicated by the arrow in FIG. 6. As the packaging 1 moves forward, the enlarged claws on the fingers 3 are forced radially inwards by the action of the cam surfaces 6 entering boss 9 in the front housing 10 of the autoinjector. This causes the blades 5 to engage the rubber needle sheath 7 even more tightly. This gripping separates the needle sheath 7 from the needle 8 as the packaging 1 is removed.

The fingers 3 are radially flexible and this is important to permit installation of the syringe in the autoinjector during initial assembly. The radial flexibility of the fingers 3 also ensures a good grip on the rubber needle sheath 7 during removal of the packaging.

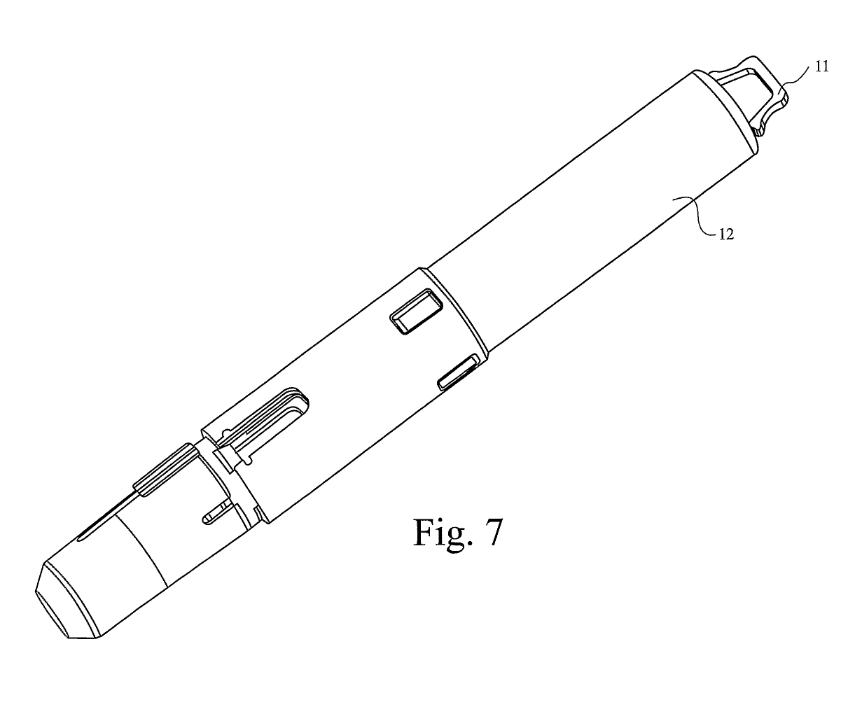

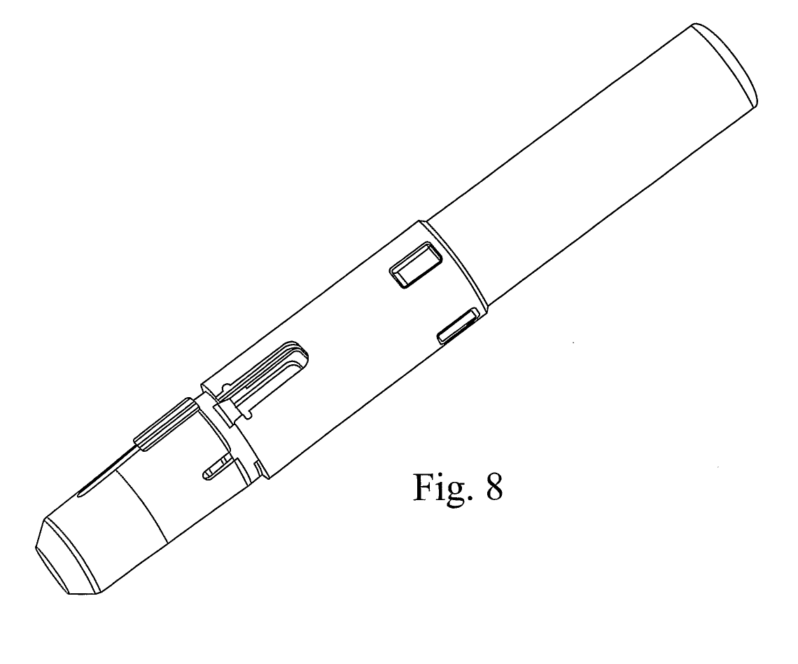

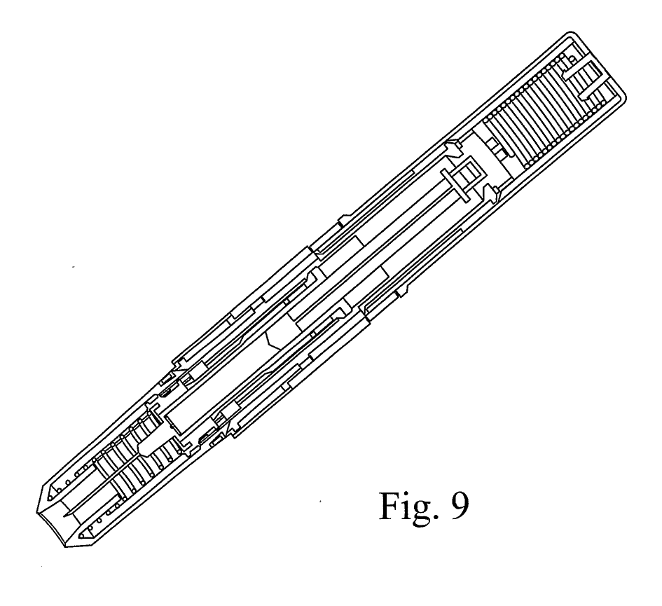

FIG. 7 shows the autoinjector removed from the packaging 1, 2 but with a safety clip 11 in place. This safety clip 11 may be a standard “transit clip” normally used to protect the autoinjector from damage when in transit in a partially-assembled condition before delivery to the end user. The purpose of the transit clip is to prevent the rear sub-assembly from firing before it is assembled to the front sub-assembly (which includes the front end cap) and until the two sub-assemblies are assembled together with the medicament-containing syringe. Once fully assembled, the front end cap 1 takes over as the means of preventing inadvertent firing of the autoinjector. The usual function of the transit clip 11 is to prevent relative movement between the outer housing 12 and the rear housing 13 of an ASI autoinjector (see FIG. 2) but here it serves the additional purpose of providing the user with the familiarity of removing a safety clip from the rear of the autoinjector before actuating delivery of the medicament. The safety clip 11 can be removed by pulling it axially rearwardly away from the autoinjector, leaving the autoinjector in the condition illustrated in FIGS. 8 and 9, wherein the autoinjector can be actuated in the normal fashion by providing a forward force to the autoinjector when placed at the injection site. Operation of the autoinjector to deliver medicament is known and not within the scope of the present invention.

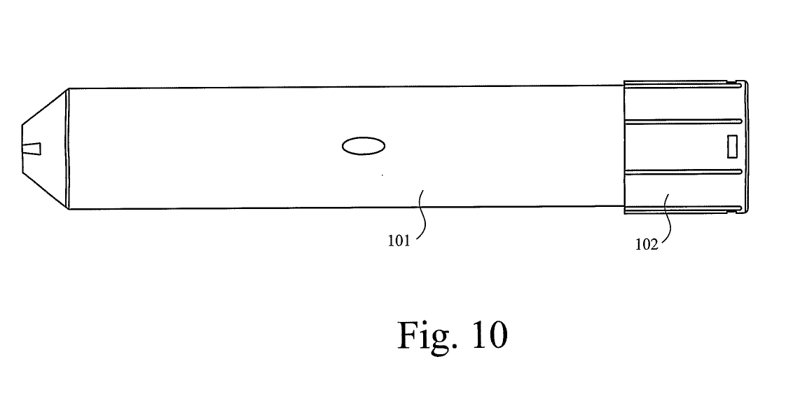

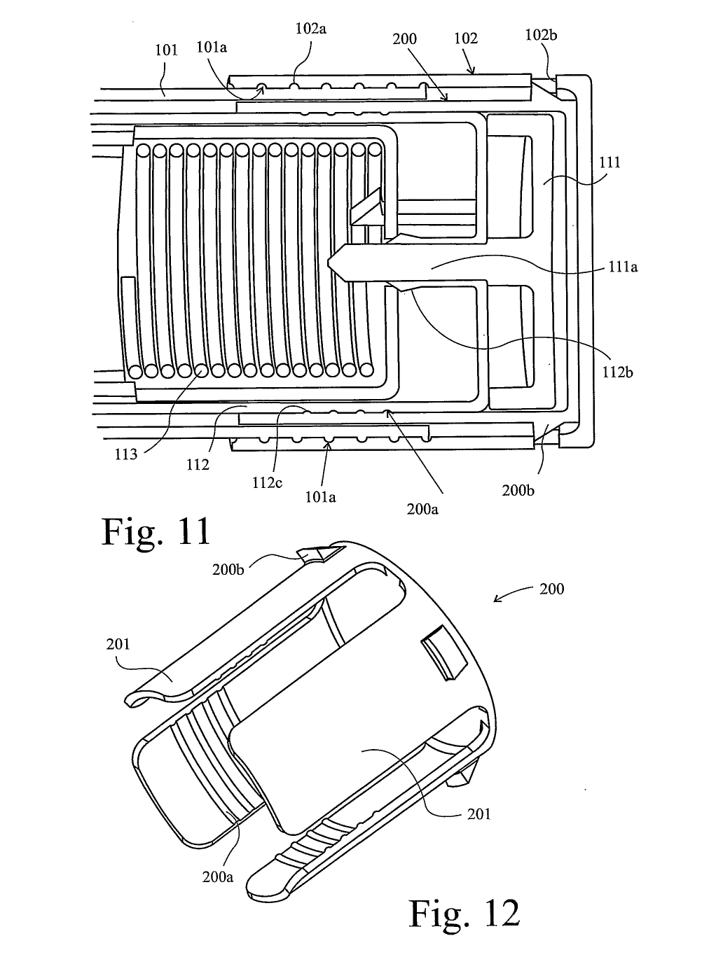

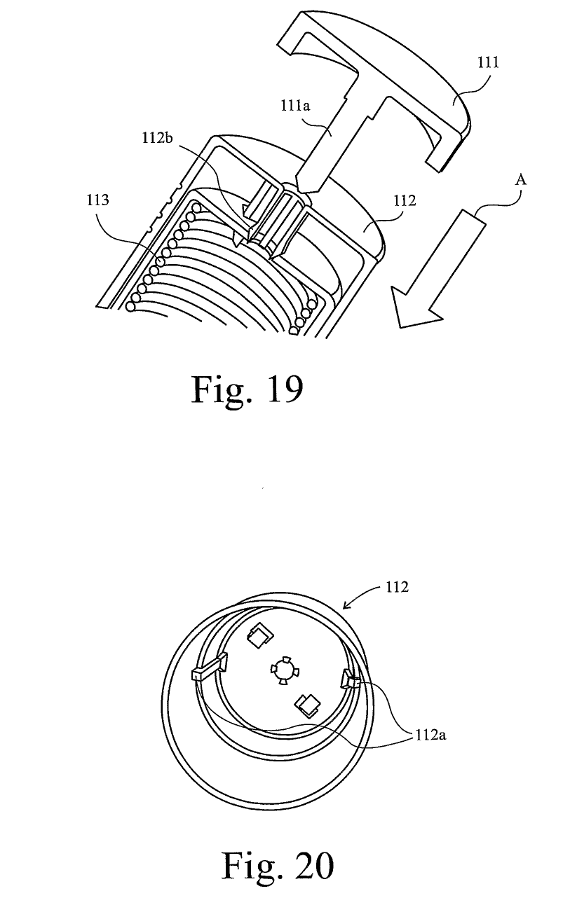

An alternative embodiment of the present invention is shown in FIGS. 10 to 20. In particular, FIG. 10 shows an alternative autoinjector including a two-part outer packaging 101, 102. Like the forward part 1 of the outer packaging described above, the forward part 101 of the outer packaging of FIG. 10 is an extended version of the front end cap of the ASI autoinjector described in WO 2005/070481 et al and continues to serve the function of preventing premature actuation of the autoinjector in the manner described in our earlier patent applications. As shown in FIG. 11, the rear part 102 of the outer packaging is generally cylindrical and has a female screw thread 102a and is screw threaded on a male screw thread 101a at a rear end of the forward part 101 of the outer packaging. When the rear part 102 of the outer packaging is screwed onto the forward part 101 of the outer packaging, the medicament-delivering part of the autoinjector is preferably entirely contained and protected. In an alternative embodiment, the rear part 102 may have a male screw thread whilst the forward part 101 may have a female screw thread.

A cap insert 200 is retained within the rear part 102 of the outer packaging such that the cap insert 200 is axially and rotationally fixed within the rear part 102. In particular, in the embodiment shown in the Figures, the cap insert 200 has radial protrusions 200b that form a snap fit with apertures 102b of the rear part 102. The cap insert 200 has circumferential ribs 200a on an inner surface that are engageable with circumferential grooves 112c of the outer housing 112 of the device. The cap insert 200 is shown in further detail in FIG. 12 where it is shown to be generally cylindrical with a plurality of axial slots defining a plurality of axially extending panels 201 that are discontinuous about the circumference of the cap insert 200.

When the rear part 102 is screwed on the screw thread 101a of the forward part 101, the panels 201 of the cap insert 200 are prevented from flexing radially by abutment with an inner surface of the forward part 101 of the outer packaging. Consequently, in the position shown in FIG. 11, relative axial movement between the cap insert 200 and the outer housing 112 is prevented by engagement between the ribs 200a and the grooves 112c.

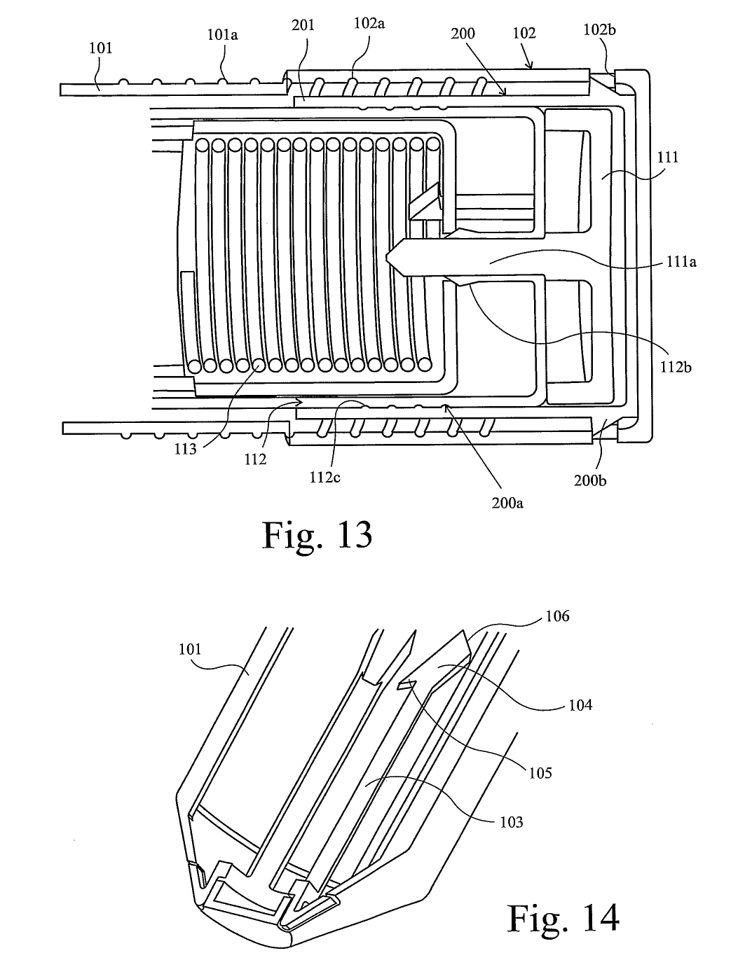

The rear part 102 of the outer packaging may be screwed relative to the forward part 101 to the position shown in FIG. 13. In this position, the cap insert is no longer in radial alignment with the forward part 101 of the outer packaging so that the panels 201 are no longer radially restrained. Upon the application of a suitable rearward force (e.g. a rearward pulling action on the rear part 102), the ribs 200a disengage from the grooves 112c and the panels 201 flex radially outwardly so that the rear part 102 and the cap insert 200 can be removed entirely from the device.

The act of unscrewing the rear part 102 of the outer packaging from the forward part 101 of the outer packaging causes the internal components of the device to move axially rearwardly relative to the forward part 101 of the outer packaging (due to the engagement between the ribs 200a and the grooves 112c). As shown in FIG. 14, the interior front end of the forward part 101 of the outer packaging is provided with a plurality of inwardly axially extending fingers 103 each having an enlarged head or “claw” 104 which is capable of gripping radially inwardly on the rubber needle sheath 107 as shown in FIG. 16. Each claw 104 has an angled blade 105 on its inner surface, and a cam surface 106 on its outer surface. In the embodiment shown in FIG. 14, the fingers 103 are attached to the forward part 101 of the packaging by a snap fit connection, although in alternative embodiments, the fingers 103 may be integrally formed with the forward part 101 of the packaging.

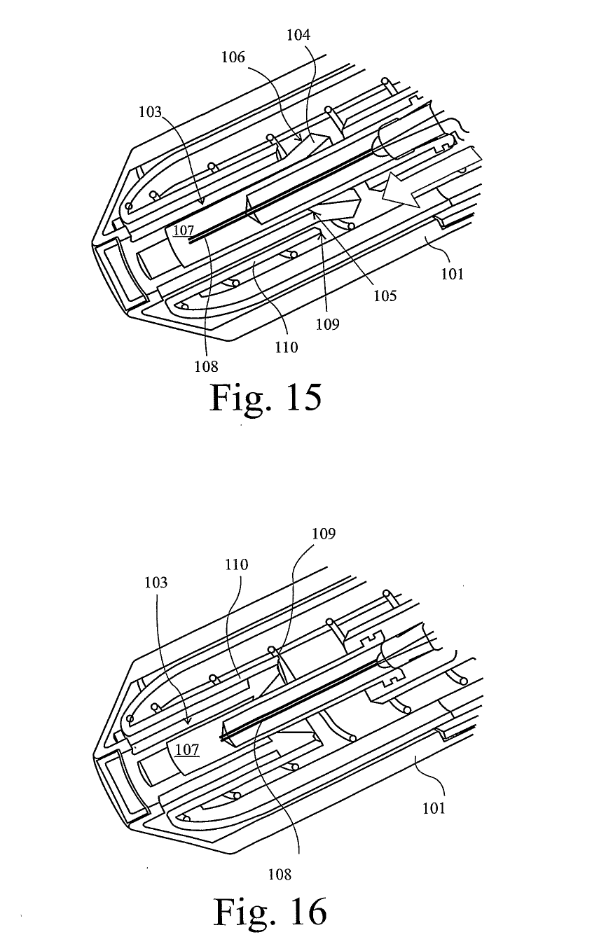

As shown in FIG. 15, before the rear part 102 of the packaging is screwed off the forward part 101, the blades 105 are engaged in the rubber needle sheath 107 which surrounds the needle 108 of the standard syringe contained within the autoinjector. When the user unscrews the rear part 102 of the packaging from the forward part 101 of the packaging, the forward part 101 of the packaging moves relative to the needle sheath 107. This relative movement causes the claws 104 on the fingers 103 to be forced radially inwards by the action of the cam surfaces 106 entering boss 109 in the front housing 110 of the autoinjector. The blades 105 then engage the rubber needle sheath even more tightly (FIG. 16). As the forward part 101 of the packaging moves axially forward further relative to the autoinjector, the gripping causes the needle sheath 107 to separate entirely from the needle 108.

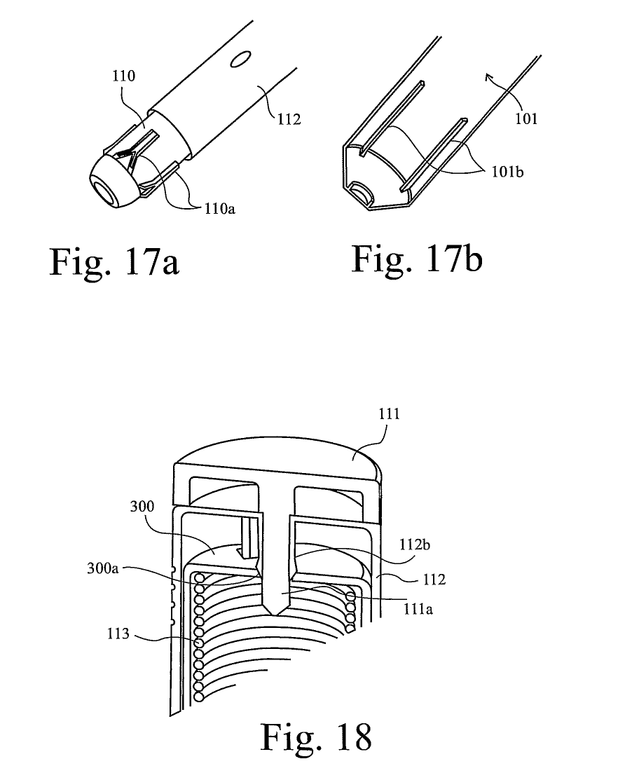

Once the rear part 102 of the packaging has been unscrewed and removed from the forward part 101 of the packaging, the needle sheath 107 is free from the needle 108 such that the device can be easily removed from the forward part 101 of the packaging by inverting or tipping the forward part 101. During removal of the rear part 102 of the packaging, the device is prevented from rotating relative to the forward part 101 of the packaging by engagement between axial ribs 101b on an inner surface of the forward part 101 and axial ribs 110a of the front housing 110 (see FIGS. 17A and 17B). Therefore, potentially damaging torque is not transferred from the unscrewing process to the needle 108 and other components of the device. Of course, any axial groove/rib arrangement that is capable of achieving this effect may alternatively be used in alternative embodiments. Additionally, during removal of the rear part 102 of the packaging, the syringe is prevented from being pulled forward by a blocking mechanism (not shown).

With the packaging 101, 102 removed, the device is prevented from actuating by the safety clip 111. As shown in FIG. 18, the safety clip 111 has an axially extending central spigot 111a that is positioned between a plurality (preferably four) of flexible legs 112b that each extend axially from the inside of the rear end of the outer housing 112. Each flexible leg 112b has a cam surface on its outer radial surface and is partially positioned inside an aperture 300a in the rear end of a spring housing 300. In a relaxed state, with the spigot 11a between them, the legs 112b have a combined diameter that is larger than the diameter of the aperture 300a and so are unable to pass through the aperture 300a. This interference prevents actuation of the device. In order to actuate the device, the user removes the safety clip 111 thereby removing the spigot 111a from its position between the flexible legs 112b. In the absence of the spigot 111a, interference between the cam surfaces of the legs 112b and the aperture 300a cause the legs 112b to flex radially inwardly and pass through the aperture 300a as the outer housing 112 is moved axially forwards relative to the spring housing 113. This action will occur when the autoinjector is placed at an injection site and a forward force is applied to the outer housing 112. Operation of the autoinjector to deliver medicament is known and is not within the scope of the present invention.

Using the improved autoinjector of the present invention, the user is able to prepare the autoinjector for delivery of medicament using familiar method steps.

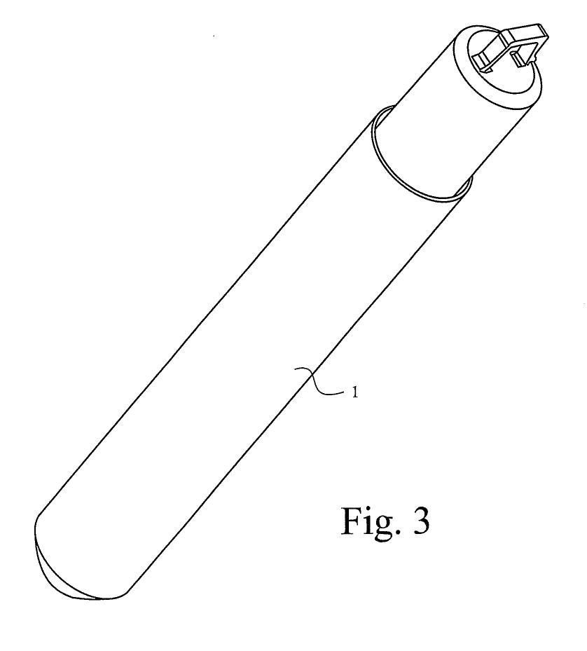

Comparable with method step 1 for a conventional epinephrine autoinjector, the first step for the user is to remove the rear part 2, 102 of the outer packaging, as shown in FIGS. 3 and 13.

Comparable with method step 2 for a conventional epinephrine autoinjector, the second step for the user is to remove the medicament-delivering part of the autoinjector from the forward part 1 of the outer packaging. In the first described embodiment, this is done by pulling or otherwise withdrawing the medicament-delivering part of the autoinjector from the forward part 1 of the outer packaging. As this happens, the rubber needle sheath 7 is pulled from the needle 8 by the internal blades 5 of the forward part 1. A spring or other means may be provided to automatically disengage the rubber needle sheath from the needle so that the autoinjector can be simply tipped out of forward part 1. In the second described embodiment, the medicament-delivering part of the autoinjector is removed from the forward part 101 of the outer packaging by a combination of unscrewing the rear part 102 and then pulling or otherwise withdrawing the medicament-delivering part of the autoinjector. The rubber needle sheath 107 is automatically pulled from the needle 108 by the internal blades 105 of the forward part 101 as the rear part 102 is unscrewed.

Comparable with method step 3 for a conventional epinephrine autoinjector, the third step for the user is to remove the “safety clip” 11, 111 or transit clip from the rear end 2, 102 of the autoinjector.

Comparable with method step 4 for a conventional epinephrine autoinjector, the fourth step for the user is to push the autoinjector onto the injection site in the usual way to actuate delivery of medicament.

Comparable with method step 5 for a conventional epinephrine autoinjector, the final step for the user is to wait the requisite period of time before removing the autoinjector from the injection site. Advantageously, the needle will have been automatically retracted into the housing and will not be visible or accessible to the user after injection.

It can therefore be seen that the improved ASI autoinjector is able to emulate the user-operated method steps of a conventional epinephrine autoinjector despite having an entirely different internal construction and operative mechanism for delivering medicament and engaging and retracting the needle.

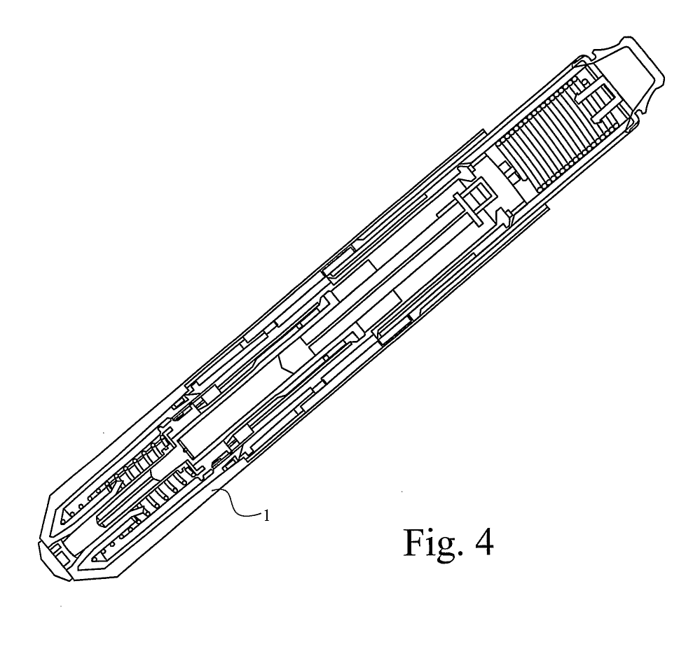

The skilled reader will appreciate that any non-mutually exclusive features of the embodiments of FIGS. 1 to 9 and 10 to 20 respectively may be interchanged.

It will be appreciated that the application of the present invention is not restricted to autoinjectors for epinephrine, or indeed even to ASI autoinjectors. It can equally well be used in any autoinjector where it is necessary to remove a needle sheath from the needle contained therein prior to actuation of the autoinjector to deliver medicament.

1. An autoinjector device for delivering a dose of medicament including:
a syringe including a needle wherein, prior to use of the device, the needle is substantially covered by a needle sheath;
a housing in which said syringe is located;
an outer packaging in which said housing is located, the outer packaging comprising a front packaging part and a rear packaging part; and
needle sheath removing means associated with said front packaging part,
wherein, prior to use of the device, said front and rear packaging parts cooperate together to entirely enclose said housing, syringe and needle sheath, and
wherein, upon axial separation of said front and rear packaging parts, said needle sheath removing means is capable of causing axial separation of said needle sheath from said needle so as to remove said needle sheath therefrom.

2. The autoinjector device according to claim 1, wherein said housing and said front packaging part include axial guide elements that cooperate to limit the degree of relative rotation therebetween.

3. The autoinjector device according to claim 2, wherein said axial guide elements include axial ribs on one or both of said housing and said front packaging part.

4. The autoinjector device according to claim 1, wherein said needle sheath removing means includes gripping elements that are capable of radially gripping said needle sheath.

5. The autoinjector device according to claim 4, wherein said gripping elements include a plurality of radially moveable, axially extending fingers, each of said plurality of fingers having an enlarged head capable of radially gripping said needle sheath.

6. The autoinjector according to claim 5, wherein each of said enlarged heads comprises a cam surface on a radially outward surface and said housing comprises an axially extending boss positioned to interfere with said cam surfaces during axial separation, wherein interference of said cam surfaces with said boss causes said plurality of fingers to flex further radially inwardly to grip said needle sheath,

7. The autoinjector device according to claim 1, further comprising a priming means which is required to be primed in order to ready the device for delivery of medicament.

8. The autoinjector device according to claim 7, wherein said priming means is a safety clip that prevents actuation and said priming involves removing the safety clip from the device.

9. The autoinjector according to claim 8, wherein said safety clip is attachable to a rear end of said housing and comprises an axially extending spigot that is capable of interfering with a trigger mechanism of the device to prevent actuation.

10. The autoinjector device according to claim 7, wherein said priming means is an actuation button, and said priming involves pressing said button.

11. The autoinjector device according to claim 1, wherein said axial separation involves the axial pulling of said front packaging part relative to said rear packaging part.

12. The autoinjector device according to claim 11, wherein said front packaging part is releasably attachable to said rear packaging part by push fit or snap fit engagement.

13. The autoinjector device according to claim 1, wherein said front packaging part and said rear packaging part have complementary screw threads and, prior to use, said front and rear packaging parts cooperate together by engagement of said complementary screw threads, wherein said axial separation involves the unscrewing of said front packaging part relative to said rear packaging part.

14. The autoinjector device according to claim 13, wherein said housing is axially and rotationally fixable with respect to said rear packaging part during axial separation of said front packaging part relative to said rear packaging part up to an axial separation threshold.

15. The autoinjector device according to claim 14, further comprising a cap insert that is axially and rotationally fixable relative to said rear packaging part, wherein said housing is axially and rotationally fixable with respect to said rear packaging part up to said axial separation threshold by engagement of said housing with said cap insert.

16. The autoinjector device according to claim 15, wherein said cap insert is arranged in said rear packaging part to define an annular gap that is adapted to receive a portion of the front packaging part;
wherein, during axial separation of said front packaging part and said rear packaging part up to said axial separation threshold, said cap insert is radially engagable with said housing to prevent relative axial displacement between said housing and said cap insert, and said portion of said front packaging part is capable of preventing radial disengagement of the cap insert from the housing;
and, during axial separation of said front packaging part relative to said rear packaging part beyond said axial separation threshold, said cap insert is radially disengageable from said housing.

17. The autoinjector device according to claim 16, wherein said cap insert comprises radially flexible panels each having a plurality of circumferential ribs projecting radially inwardly therefrom, wherein the plurality of ribs are engageable with a plurality of circumferential grooves on said housing.

18. A method of preparing an autoinjector device for delivering a dose of medicament comprising the steps of:
providing an autoinjector device, including:
a syringe including a needle wherein, prior to use of the device, the needle is substantially covered by a needle sheath;
a housing in which said syringe is located;
an outer packaging in which said housing is located, the outer packaging comprising a front packaging part and a rear packaging part; and
needle sheath removing means associated with said front packaging part,
wherein, prior to use of the device, said front and rear packaging parts cooperate together to entirely enclose said housing, syringe and needle sheath;
axially separating said front and rear packaging parts, causing said needle sheath to be removed from said needle; and
removing said front and rear packaging parts from the device; such that the autoinjector device is ready to deliver a dose of medicament.

19. The method according to claim 18, wherein the provided autoinjector device further includes a priming means which is required to be primed in order to ready the device for delivery of medicament, and the method further comprises the step of priming the device using said priming means such that autoinjector device is ready to deliver a dose of medicament.

20. A The method according to claim 19, wherein said axial separation involves the axial pulling of said front packaging part relative to said rear packaging part, and wherein the step of axially separating said front and rear packaging parts includes axially pulling the front packaging forwardly relative to said rear packaging part, consequently causing said needle sheath to be removed from said needle.

21. The method according to claim 19, wherein said front packaging part and said rear packaging part have complementary screw threads and, prior to use, said front and rear packaging parts cooperate together by engagement of said complementary screw threads, wherein said axial separation involves the unscrewing of said front packaging part relative to said rear packaging part, and wherein the step of axially separating said front and rear packaging parts includes unscrewing said front packaging part from said rear packaging part, consequently causing said needle sheath to be removed from said needle.

22. The method according to claim 21,
wherein said cap insert is arranged in said rear packaging part to define an annular gap that is adapted to receive a portion of the front packaging part;
wherein, during axial separation of said front packaging part and said rear packaging part up to said axial separation threshold, said cap insert is radially engagable with said housing to prevent relative axial displacement between said housing and said cap insert, and said portion of said front packaging part is capable of preventing radial disengagement of the cap insert from the housing;
and, during axial separation of said front packaging part relative to said rear packaging part beyond said ax separation threshold, said cap insert is radially disengageable from said housing,
and wherein the step of axially separating said front and rear packaging parts includes unscrewing said front packaging part from said rear packaging part at least up to said axial separation threshold, and then axially pulling said front packaging part from said rear packaging part.

21-24. (canceled)
